(19) United States
(12) Patent Application Publication
(10) Pub. No.: US 2024/0159773 A1
(43) Pub. Date: May 16, 2024

(54) Method of Evaluating Efficacy of Chiropractic Care for Chronic Lower Back Patients

(71) Applicants: Maria Arantzazu Ortega De Mues, Madrid (ES); Ricardo Fujikawa Kawahara, Madrid (ES); Kean J. DeCarlo, Salem, SC (US)

(72) Inventors: Maria Arantzazu Ortega De Mues, Madrid (ES); Ricardo Fujikawa Kawahara, Madrid (ES); Kean J. DeCarlo, Salem, SC (US)

(21) Appl. No.: 18/511,323

(22) Filed: Nov. 16, 2023

Related U.S. Application Data
(60) Provisional application No. 63/425,860, filed on Nov. 16, 2022.
(60) Provisional application No. 63/441,497, filed on Jan. 27, 2023.

(51) Int. Cl. G01N 33/68

(52) U.S. Cl. CPC G01N 33/6863; G01N 2800/52

(57) ABSTRACT

A method and kits for chiropractic treatment protocols to be administered to a patient that includes obtaining a urine sample from the patient; measuring the level of TGF-α cytokine thereof in the normalized urine sample; comparing the level of the TGF-α cytokine to a minimum threshold level in patients suffering from chronic lower back pain; and administering desired protocols of chiropractic treatment to the patient on the comparison of TGF-α cytokine levels.

### CROSS-REFERENCE TO RELATED APPLICATIONS

This application claims benefit of priority from U.S. Provisional Application No. 63/425,860, titled “Method of Evaluating Efficacy of Chiropractic Care for Chronic Lower Back Patients,” which was filed Nov. 16, 2022, and U.S. Provisional Application No. 63/441,497, titled “Method of Evaluating Efficacy of Chiropractic Care for Chronic Lower Back Patients,” which was filed Jan. 27, 2023, which applications are hereby incorporated by reference.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present disclosure relates generally to systems, kits, and methods in the field of pain management and treatment, more particularly, to various aspects involving systems, kits and methods for improved chiropractic outcomes using a method and kit that utilizes the identification of a heightened level of the inflammatory cytokine Tumor Necrosis Factor-alpha (TNF-α) in the urine of the patient.

### BACKGROUND

Pain, and particularly, low-back pain, is a leading cause of disability worldwide. Lower back pain can be considered chronic is it last three months or more and can reappear intermittently, causing frustration to the afflicted patient. Lower back pain can be age related or can result from prior injury. The most common causes include arthritis of the spine, resulting in the gradual thinning of the cartilage inside the spine; spinal stenosis, resulting in the narrowing of the spinal canal that may lead to nerve pain; herniated or bulging discs; and myofascial pain syndrome, resulting in unexplained muscle pain and tenderness.

Most cases of lower back pain are described as being non-specific, implying that the etiology of the patient's complaints is unknown and cannot be attributed to a single tissue or pathology. A growing body of research is being directed toward identifying biomarkers and biopsychosocial risk factors that influence the course of the disease.

One symptomatic corollary to lower back pain is an increased level of inflammatory response. A variety of inflammatory cytokines profiles and biochemical markers have been linked to different stages of non-specific lower back pain.

Clinical research and animal models suggest that spinal manipulation might modulate plasma levels of inflammatory cytokines, which have been involved in different stages of low back pain. More specifically, serum levels of the inflammatory cytokine Tumor Necrosis Factor-alpha (TNF-α) have been found to be elevated in patients with chronic low back pain. Clinical research and animal models suggest that spinal manipulation can be effective to modulate plasma levels of inflammatory cytokines,—including TNF-α, which are in different stages of low back pain.

Typically, blood plasma from a patient is the preferred medium for testing the levels of TNF-α. However, this testing is invasive and typically requires a multi-day lab protocol to obtain the final lab results. Hence, there is need for the development of minimally invasive procedures for a biological sample retrieval suitable for rapid detection of patient TNF-α levels.

The present invention provides methods and compositions for detection of patient TNF-α levels and diagnosis of diseases based on this detection.

Accordingly, there is a need for continued improvement in the use and operability of non-invasive testing of a patient to determine heightened levels of TNF-α in a patient. There is a particular need for improvement in the use and operability of non-invasive testing of a patient to determine, on a timely basis, heightened levels of TNF-α in a patient undergoing various chiropractic treatments where it is desirable to monitor patient lower back pain recovery progress over the course of the chiropractic treatments.

### SUMMARY

To improve the state of the art, disclosed herein is a non-invasive testing protocol for determining heightened TNF-α levels in a patient, and kits for use therein, utilizing novel features and functionalities. In one embodiment, a method and kit is provided for detecting TNF-α levels in a patient from a urine specimen to detect a TNF-α levels in the patient that is associated with a disease or condition.

In a further embodiment, a non-invasive testing protocol is provided to detect TNF-α levels in a patient from a urine specimen that is associated with the level of inflammatory response in the patient and which is associated with the likelihood of occurrence of a disease or condition.

A growing body of research has linked a variety of inflammatory cytokines profiles and biochemical markers to different stages of non-specific lower back pain. These cytokine profiles are predominantly quantified through serum analysis although accumulating evidence suggests that it may be possible to detect some of these biomarkers in urine with a high degree of correlation with circulating levels. Thus, urinalysis could provide a cheaper, non-invasive alternative to blood samples that is much easier to collect in a private or smaller clinical setting.

Analysis of and respective levels of the inflammatory cytokine Tumor Necrosis Factor-alpha (TNF-α) can aid in the pathogenesis and in possible treatment strategies of chronic lower back pain. TNF-α is a potent pro-inflammatory cytokine that was closely associated with intervertebral disk degeneration, though it may also induce a downstream cascade of other cytokines involved in chronic lower back pain such as interleukin-6 or interleukin-1β. Observational studies have found elevated serum levels of TNF-α in patients with severe sciatica and chronic lower back pain compared to patients with milder symptoms or healthy controls. The in vitro production of TNF-α has been found to be increased (and positively correlated to that of interleukin 16) in chronic lower back pain when compared to healthy control and acute lower back pain individuals.

Chiropractic care offers a conservative option for the management of chronic lower back pain. To address this condition, chiropractors use a multimodal approach, including manual therapy along with exercise and patient education as part of a toolkit of routine interventions. However, spinal manipulative therapy (SMT) is the main intervention utilized in chiropractic practice. SMT has been found to be as effective for chronic lower back pain as other recommended interventions, such as exercise therapy and is currently recommended in the latest clinical practice guidelines. For both manipulative and exercise therapy, multiple potential mechanisms for pain relief have been proposed. The specific mechanisms are still unknown, but human and animal studies have shown a correlation between SMT and a decrease in serum levels and in vitro production of pro-inflammatory mediators, including TNF-α. Similarly, investigations on the mechanisms of exercise therapy for chronic lower back pain have also found that a reduction in levels of the same cytokine network may be involved in pain relief associated to exercise therapy.

In one aspect, the treatment protocol includes the determination of the presence of, and respective level of, TNF-α in urine samples of chronic lower back pain patients, and to assess the relative efficacy of a period of multimodal chiropractic care mainly based in SMT, in parallel to pain intensity and disability, based on the tested levels of this cytokine that are obtained over the course of the treatment period of the multimodal chiropractic care.

Still other aspects, embodiments, and advantages of these exemplary aspects and embodiments, are discussed in detail below. Moreover, it is to be understood that both the foregoing information and the following detailed description are merely illustrative examples of various aspects and embodiments and are intended to provide an overview or framework for understanding the nature and character of the claimed aspects and embodiments. Accordingly, these and other objects, along with advantages and features of the present invention herein disclosed, will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain the principles of the embodiments discussed herein. No attempt is made to show details of this disclosure in more detail than can be necessary for a fundamental understanding of the exemplary embodiments discussed herein and the various ways in which they can be practiced.

FIG. 1 schematically illustrates the investigatory study protocol, including outcomes measures collected in relationship to the treatment period.

FIG. 2 graphically illustrates results of the investigatory study protocol. Urinary TNF-α levels in chronic low back pain (CLBP) patients, pre- and post-treatment. For each sample, the urinary concentrations of TNF-α(pg/ml) and creatinine (mg/dl) were assessed. The ratio of urinary TNF-α to urinary creatinine in pg/mg was calculated to correct changes in urine volume. In FIG. 2, the middle horizontal line in the boxes represents the median and the x represents the mean, while the upper and the lower lines in each box represent the first and third quartile respectively and the whiskers include all individual data points within 1.5 times the interquartile range.

FIGS. 3A and 3B graphically illustrates clinical variables measured in chronic low back pain patients, pre- and post-treatment in the investigatory study protocol. FIG. 3A shows pain intensity scores reported in a Numerical Rating Scale, from 0 to 10, pre- and post-treatment. Similarly, FIG. 3B shows disability scores reported in the Oswestry Disability Index questionnaire, from 0 to 50, pre- and post-treatment. In FIGS. 3A and 3B, the middle horizontal line in the boxes represents the median and the x represents the mean, while the upper and the lower lines in each box represent the first and third quartile respectively and the whiskers include all individual data points within 1.5 times the interquartile range.

FIG. 4 shows participant demographic and baseline data for participants in the investigatory study.

FIG. 5 shows before and after treatment variations in TNF-α concentrations, pain intensity and disability in patients participating in the investigatory study.

FIG. 6 shows mean baseline and follow-up TNF-α values and mean differences between baseline and follow-up TNF-α values in the primary and secondary outcomes for the second investigatory study. For this figure, TNF-α values are corrected values measured in pg/mg, NRS values refer to pain intensity in the numerical rating scale from 0 to 10, and ODI values refer to disability, measured with the Oswestry Disability Index from 0 to 50.

FIG. 7 shows a violin plot of the distribution of urinary concentrations of TNF-α corrected for volume, at baseline and follow-up. Individual dots represent individual participant values for the second investigatory study. The continuous line represents the median and dotted lines represent 25th and 75th quartiles.

FIGS. 8A and 8B show violin plots of the distribution of pain intensity ratings in the numerical rating scale (NRS) from 0 to 10 (FIG. 8A) and disability scores measured with the Oswestry Disability Index (FIG. 8B), both at baseline and follow-up. Individual dots represent individual participant values for the second investigatory study. The continuous line represents the median and dotted lines represent 25th and 75th quartiles.

FIG. 9 is a schematic view of a TNF-α lateral flow detection kit showing a sample pad configured to absorbs a urine sample and to transport portions of the urine sample to a conjugate pad that contains at least one antibody-nanoparticle conjugate. Also shown is a reagent membrane having test and control lines that show the assay results and a wicking pad that is configured to pull the urine sample through the test strip at a desired rate.

FIG. 10 shows a perspective view of an exemplary schematic of a TNF-α lateral flow detection kit cassette.

FIG. 11 shows a perspective view of an exemplary sandwich assay format.

FIG. 12 shows a schematic view of the preparation of an exemplary lateral flow TNF-α lateral flow detection kit cassette.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “cytokine” can include two or more such cytokines unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “can,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular embodiment.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed but are used merely as labels to distinguish one claim element having a certain name from another element having a same name (but for use of the ordinal term) to distinguish claim elements.

The term “diagnosis” as used herein and its grammatical equivalents, means the testing of subjects to determine if they have a particular trait for use in a clinical decision. Diagnosis includes testing of subjects at risk of developing a particular disease resulting from infection by an infectious organism or a non-infectious disease, such as cancer or a metabolic disease. Diagnosis also includes testing of subjects who have developed particular symptoms to determine the cause of the symptoms, Diagnosis also includes prognosis, monitoring progress of a disease, and monitoring the efficacy of therapeutic regimens. The result of a diagnosis can be used to classify patients into groups for performance of clinical trials for administration of certain therapies.

The term “treating” and its grammatical equivalents as used herein include achieving a therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

As used in a general sense herein, unless otherwise indicated by context, the term “antibody” includes “antibody fragment.”

Disclosed are components that can be used to perform the disclosed methods and systems. These and other components are disclosed herein, and it is understood that when combinations, subsets, interactions, groups, etc. of these components are disclosed that while specific reference to each various individual and collective combinations and permutation of these cannot be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems can be understood more readily by reference to the following detailed description of preferred embodiments and the examples included therein and to the Figures and their previous and following description.

An examination of chronic low back pain patients participating in a first investigatory study underwent a customized chiropractic care plan consisting in each encounter of a case history, physical assessment and multimodal care has now shown that, surprisingly, urine from chronic low back pain patients is an appropriate medium to measure concentrations of TNF-α and to examine possible changes in its levels associated to chiropractic care.

In the first investigatory study, all patients received full-spine SMT, however, all other interventions were used less frequently, based on patient and clinician preferences, and needs. This pragmatic setting was preferred as it reflects better real clinical practice. Spinal manipulation consisted in high-velocity low amplitude thrust manipulations, applied to joints with restricted motion by hand with or without the assistance of a drop-piece mechanism. When used, exercise recommendations were based on a customized set of stretching and mobilization exercises focused on the lumbopelvic area, to be performed for less than 5 min on a daily basis. Patient education was limited to advice to remain active and reassurance about the benign nature of the patients' condition. Care was delivered by a chiropractic intern, under the supervision of a trained chiropractor with at least 5 years of clinical expertise. The duration of care and the total number of treatment sessions received per patients was not pre-established. Instead, patients were offered a treatment plan adapted to their clinical presentation, with 1 or 2 weekly visits for a range of 4 to 8 weeks. This is in accordance with recommendations from a clinical practice guideline that suggests 1-3 weekly visits for about 4 weeks for chronic low back pain.

At the end of the treatment period, a complete physical re-evaluation of the patient was performed, including evaluation of the main outcome measures, described below. Care was purposely not modified for the sake of the first investigatory study. The control group did not receive any type of treatment. The inclusion of this control group aimed at defining a reference value for urinary TNF-α in a healthy population, in order to confirm that persons with chronic low back pain have higher than normal urinary TNF-α levels. TNF-α levels are presumed to remain relatively stable, nearing zero in the absence of active or chronic inflammation. Reference ranges and biological variability over 6 weeks for serum TNF-α in healthy subjects have been reported. For this first investigatory study, a mean value of 1.51 pg/mL and the upper limits of 2.53 pg/ml (95th percentile) and 3.30 pg/mL (99th percentile) were deemed acceptable for diagnosis of systemic inflammation. Because, at the time of the first investigatory study, there are no known reference values for urinary levels of TNF-α, the control group served as a comparison group providing a reference value, which was used to test the hypothesis of abnormally high levels of TNF-α in the chronic low back pain groups when compared to a healthy population.

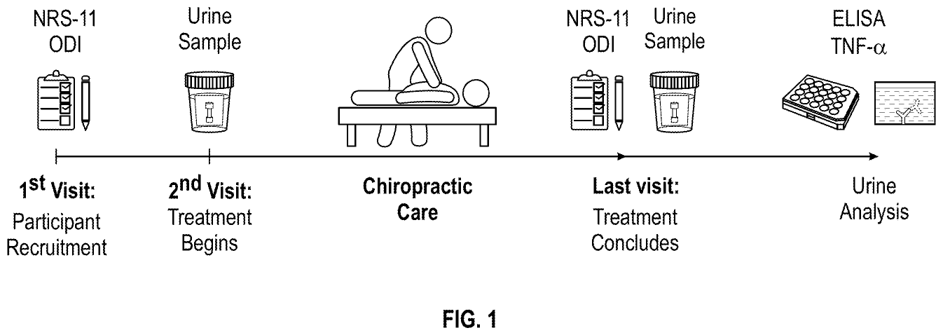

In the first investigatory study, both patients and healthy participants provided an initial urine sample (first micturition of the morning) before any treatment was initiated. Control subjects provided a unique urine sample at the beginning of the first investigatory study as the only outcome measure, since they did not receive any treatment and the levels of TNF-α did not change significantly over time in the absence of active or chronic inflammation. Referring to FIG. 1, patients collected their first urine sample on the same morning of the initial treatment session and a urine sample on the morning after the last session of the treatment period. For all cases, patients were instructed to provide the first micturition of the morning and preserve their sample refrigerated for as long as possible before their appointment in the clinic. Urine samples were then collected from patients and immediately stored in a container at −20° C. Urine concentration of TNF-α was measured in duplicate by using specific commercial sandwich enzyme-linked immunosorbent assay (ELISA) following manufacturer's recommendations (Cloud-Clone Corp., TX, United States). For each sample, the urinary concentrations of TNF-α (pg/ml) and creatinine (mg/dl) were assessed. The ratio of urinary TNF-α to urinary creatinine in pg/mg was calculated to correct changes in urine volume.

Patient-reported outcome measures (PROMs) were used to evaluate pain and disability. Both were measured at the beginning and upon completing the treatment period. Patients reported current pain intensity verbally using a Numerical Rating Scale from 0 to 10 (NRS-11), in which 0 signified the absence of pain and 10 the worst possible pain for the patient. Functional disability due to chronic low back pain was measured using the validated version of the Oswestry Disability Index (ODI). The ODI questionnaire consists of 10 multiple-choice questions rated from 0 to 5, with a total possible score of 50 (maximal disability).

In order to detect possible differences in normalized TNF-α (TNF-α to creatinine ratio) urinary concentrations between patients with chronic low back pain and healthy controls, the mean value was calculated from the latter and used as a reference value. Further, Welch's t-tests were used to compare the mean of the chronic low back pain group to this reference value, both pre- and post-treatment, as this adaptation of the t-test is known to be robust against type I errors. Urinary TNF-α concentrations before and after chiropractic treatment were later compared by using a paired t-test. Additionally, paired t-tests were used to ascertain differences in PROMs (NRS-11 and ODI scores) before and after exposure to chiropractic treatment.

In order to assess for correlations between concentrations of TNF-α, pain intensity, disability and number of visits, Spearman's rank correlation coefficient was calculated by using before and after treatment values and percent changes. Finally, the potential interaction of treatment variables other than SMT (exercise and medication use) was analyzed by comparing the mean variations in TNF-α levels (L}._{TNF-α}), NRS-11 (L}._{NRS-11}) and ODI scores (L}._{ODI}) in participants following a home exercise program or using pain medication, versus participants who did not. Differences in these variables were based on pragmatic differences in the treatment approach. Due to unequal sample sizes and variances, Welch's t-test were used for these comparisons. Values presented in the results section represent mean±standard deviation. For all before and after comparisons, effects sizes were computed and reported by means of Cohen's d. A p value threshold of 0.05 was considered statistically significant for all analyses.

In the first investigatory study, twenty-four patients (14 men and 10 women) suffering from chronic low back pain met the inclusion and exclusion criteria, were accepted to participate in the first investigatory study by providing PROMs and urine samples and concluded the treatment period. Treatment consisted of a mean of 8.4 individual visits (ranging from 3 to 13 visits), delivered during a mean of 39.4 days (ranging from 19 to 76 days). For the control group, 5 healthy subjects (3 men and 2 women) were recruited for the first investigatory study.

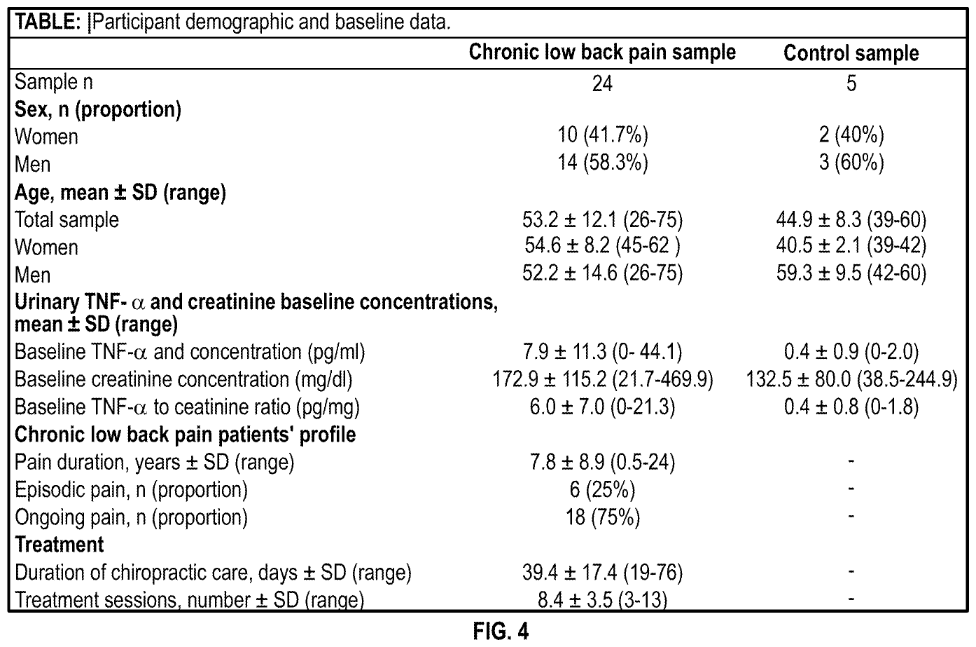

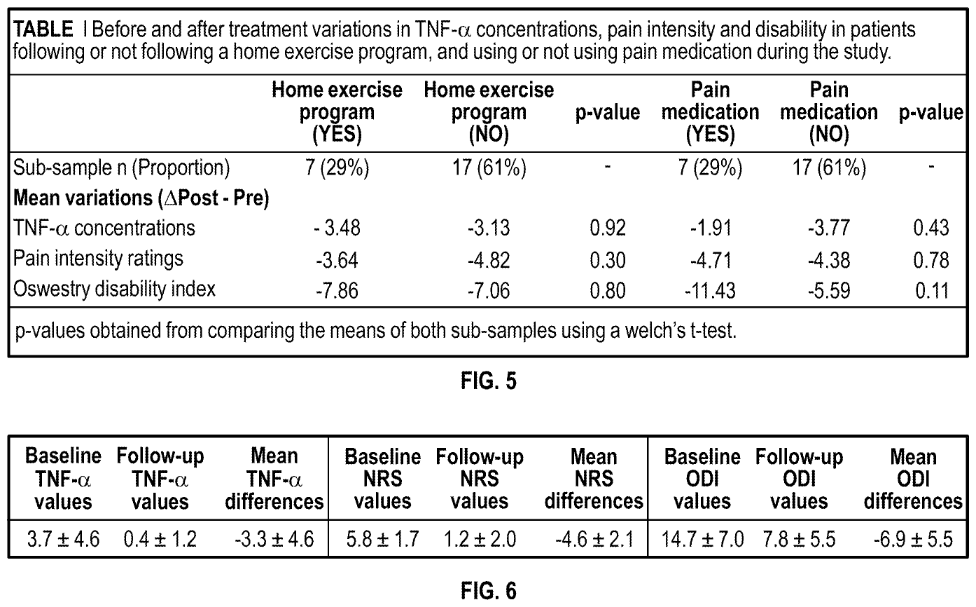

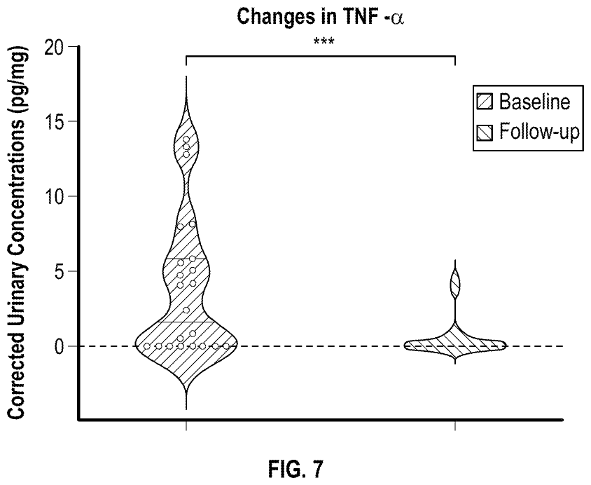

Referring to FIG. 4, the first investigatory study showed that the mean concentration of TNF-α in urinary samples of healthy participants was about 0.4±0.9 pg/ml, and, when corrected for urinary volume, the ratio to creatinine was about 0.4±0.8 pg/mg.

For the first investigatory study, participants suffering from chronic low back pain, before initiation of the treatment protocol, mean urinary TNF-α concentration was 7.9±11.3 pg/ml and the mean ratio to creatinine was 6.0±7.0 pg/mg. These levels were significantly higher when compared to the reference value obtained from healthy controls (p=0.0006). After completing the period of chiropractic care, the mean concentration of TNF-α in the urine of the patients suffering from chronic low back pain was 3.6±5.5 pg/ml, and the ratio to creatinine was 2.8±4.5 pg/mg. These levels were still significantly elevated when compared to our reference value (p=0.015).

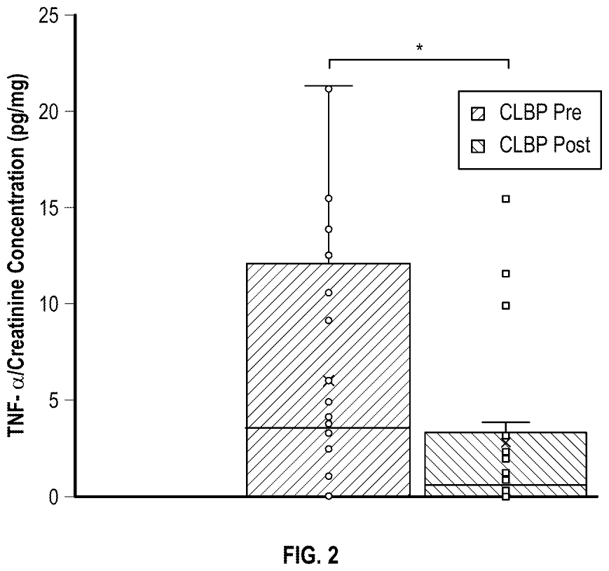

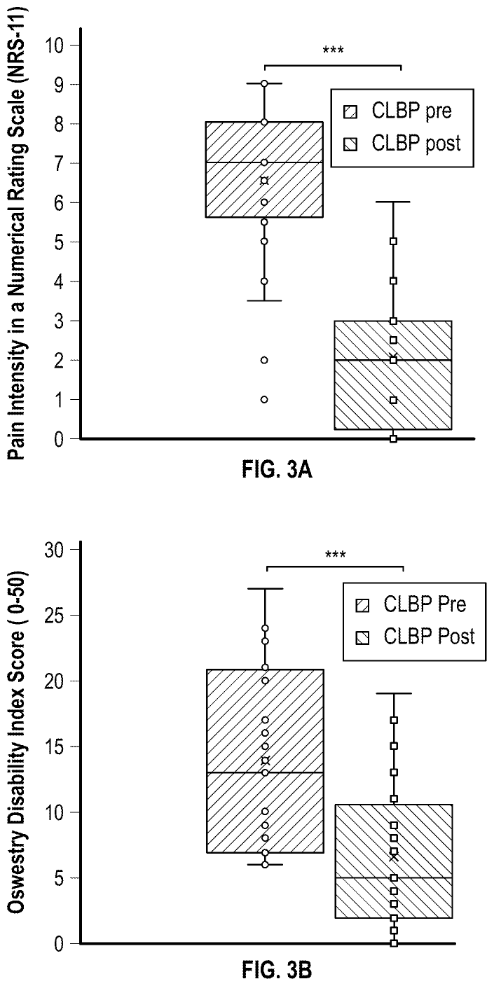

Referring to FIG. 2, it is noteworthy that urinary concentration of TNF-α corrected for volume was significantly lower after the period of chiropractic care compared to baseline (p=0.03), Cohen's d=0.55. Further, and now referring to FIGS. 3A and 3B, following the period of chiropractic care, a statistically significant reduction in pain intensity (as measured by a numerical rating scale: NRS-11) was noted for the treatment group, p<0.001, Cohen's d=2.33. Moreover, upon completing the period of chiropractic treatment, a statistically significant reduction in functional disability (as measured by the Oswestry Disability Index: ODI) was observed, p<0.001, Cohen's d=1.17.

Of interest, there was also a moderate significant correlation between the number of treatment visits and the change in NRS-11 (p=0.49, p=0.008). However, no other variable was correlated to the number of visits (change in ODI p=0.27, p=0.1; ODI post-treatment p=0.33, p=0.056; NRS-11 post-treatment p=0.32, p=0.06). No significant correlations were observed between TNF-α urinary levels at any stage or percent changes before and after treatment with any other variables analyzed.

This first investigatory study appears to be the first study to measure TNF-α levels in urine samples of chronic low back pain patients. In this cohort of chronic low back pain patients, urinary concentrations of the TNF-α cytokine were significantly elevated when compared to a reference value from healthy controls. Urinary TNF-α levels were lower after chiropractic care compared with the baseline values. This was accompanied by lower levels of pain and disability after chiropractic care compared with the levels before initiation of treatment.

In one aspect, it is contemplated that modulation of the pro-inflammatory cytokine network, such as the TNF-α cytokine, in conjunction with chiropractic care could relieve pain and decrease disability in patients with chronic low back pain. Optionally, it is further contemplated that monitoring of the level of the TNF-α cytokine could be used throughout the course of chiropractic treatment to monitor the applied efficacy of the chiropractic treatment protocol, i.e., if the level of the drops from an elevated level toward a lower, healthy level, the treating physician is reassured as to the efficacy of the treatment protocol being applied to the particular patient.

Although urinary levels of inflammatory cytokines have been measured and highly correlate to serum levels, such urinary levels of inflammatory cytokines have not been used specifically as biomarkers for chronic pain conditions. In this first investigatory study, elevated concentrations of TNF-α were discovered in urine samples of chronic low back pain patients (both before and after treatment), when compared to a healthy patient group reference value, which suggests that urine can be a reliable milieu in which biomarkers for chronic low back pain can be studied. As noted above, after the chiropractic care period, TNF-α concentrations were significantly reduced, with a medium effect size (d=0.55).

The physiological mechanism through which SMT can result in a reduction of pro-inflammatory cytokines is still unknown. Recent studies have shown that activation of pain circuits regulates neuroinflammation, both centrally and in the periphery. It is plausible that SMT-induced modulation of nociceptive input could result in a down-regulation of neuroinflammation. Such neuroimmune interactions have been extensively researched in the context of chronic pain, suggesting a bi-directional communication between nociceptive neurons and microglia necessary for the release of pro-inflammatory mediators such as TNF-α. Furthermore, central release of TNF-α seems to be required to induce long-term potentiation and central sensitization in the dorsal horn of the spinal cord, inducing persistent pain. Similarly, in both animal models and patients with rheumatoid arthritis or discogenic LBP, TNF-α blocking drugs have been shown not only to reverse central pain responses but also to improve disability. Research has also suggested that SMT could have a non-specific effect on sympathetic nervous system (SNS) function, which could in turn modulate immune responses. Involvement of the SNS in the modulation of TNF-α production and release by macrophages has been observed in animal models.

This first investigatory study suggests that SMT is an effective approach to reduce pain levels and improve function in chronic low back pain patients. As discussed, the levels of pain intensity and disability were both significantly reduced after the treatment period, although these effects were not compared with a placebo. Nevertheless, the changes observed had very large effect sizes (d=2.33 and 1.17 for pain and disability respectively), which could also be considered clinically significant. Indeed, pain intensity ratings showed a 68% reduction in pain intensity from baseline, largely exceeding the proposed threshold of 30%, while disability scores were 14.6 points lower (out of 100) after treatment, also superior to the estimated minimal detectable change for the ODI of 11.75 points. The first investigatory study identified moderate significant correlations between pain intensity levels and disability scores percentage of change, which is consistent with the ODI being an adequate tool to measure LBP-related disability.

It is reasonable to conclude, based on the first investigatory study results that urinary levels of TNF-α can be found elevated in patients with chronic low back pain when compared to healthy individuals. The results obtained in this study suggest that a non-standardized period of chiropractic care can have a significant impact on pain and disability levels, as well as on urinary concentrations of pro-inflammatory cytokine TNF-α in chronic low back pain patients.

In a further aspect, it is contemplated that detection of urinary TNF-α concentrations can be used as a prognostic biomarker for poor recovery from low back pain episodes and the development of ongoing chronic low back pain trajectories. Such use would allow for the identification of patients suffering ongoing chronic low back pain versus patients suffering from episodic low back pain, i.e., allowing for the identification of patients with trajectories described as ongoing showing greater levels of TNF-α, when compared to patients with episodic low back pain.

A second investigatory study was conducted to measure urinary levels of TNF-α in a cohort of patients with chronic low back pain, before and after a period of standardized SMT and to assess urinary TNF-α levels that could be associated with and differ according to clinical or demographic variables. The second investigatory study further confirms that measured urinary TNF-α concentrations could predict clinical outcomes after receiving SMT and represents a measurable biomarker that is suited to discriminate patients with low back pain, either chronic or episodic, according to their pain trajectory and to predict clinical recovery. This second investigatory study was an observational study, and, without a control group, no change can be attributed to the intervention or to any other factor.

In the second investigatory study, all patients underwent a standardized unimodal care plan based exclusively on the delivery of instrument assisted SMT by a chiropractor, twice a week for a total duration of four weeks. The use of an instrument assisted protocol of SMT was preferred in order to standardize treatment protocols and reduce variability in force application. Frequency of care was standardized in order to reflect clinical practice and comply with clinical practice guidelines. In this second study, instrument assisted SMT treatment provided delivery of high-velocity low amplitude manipulations with the assistance of the Activator IV mechanical device (FDA approval #K003185, Manufacturer: Activator Methods International Ltd., Phoenix, AZ). This instrument is a hand-held device containing a spring-loaded mechanism that delivers a mechanical impulse with four different settings.

At the end of the treatment period, a complete physical re-evaluation of the patient was performed, including evaluation of the main outcome measures, described below. Care was purposely not modified for the sake of the second investigatory study.

In the second investigatory study, patients provided a baseline urine sample (first micturition of the morning) before any SNT treatment was initiated. Patients collected their first urine sample on the same morning of the initial treatment session and a urine sample on the morning after the last session of the treatment period. For all cases, patients were instructed to provide the first micturition of the morning and preserve their sample refrigerated for as long as possible before their appointment in the clinic. Urine samples were then collected from patients and immediately stored in a container at −20° C. The collection procedure was identical for the follow-up sample, which was collected the day after the eighth and final session. Participants were requested to refrain from taking any anti-inflammatory medication within 24 hours of the dates when both samples were collected. Identical to process of the first investigatory study, urine concentration of TNF-α was measured in duplicate by using specific commercial sandwich enzyme-linked immunosorbent assay (ELISA) following manufacturer's recommendations (Cloud-Clone Corp., TX, United States). For each sample, the urinary concentrations of TNF-α (pg/ml) and creatinine (mg/dl) were assessed. The ratio of urinary TNF-α to urinary creatinine in pg/mg was calculated to correct changes in urine volume.

Patient-reported outcome measures (PROMs) were used to evaluate pain and disability. Both were measured at the beginning and upon completing the treatment period. Clinical variables describing comorbidities, CLBP duration and trajectories were collected in the initial clinical interview. The presence of comorbidities included chronic non-painful conditions and pain affecting other body sites. Duration since the onset of the first episode was recorded in years. CLBP trajectories were classified as either ‘ongoing,’ ‘fluctuating’ or ‘episodic’ (independent of severity), according to suggested criteria. Episodic CLBP was defined as pain occurring with pain-free periods of at least 4 weeks. The trajectory was classified as fluctuating when patients recalled variations of 2 or more points in an 11-point numerical rating scale (NRS), without 4-weeks pain-free periods. Finally, ongoing pain implied a relatively stable pain intensity (±1 point in the NRS) present seven days a week, which was more stringent than the suggested criteria of at least 4 days per week. These variables were used to identify potential patient subgroups with different levels in urinary TNF-α.

Statistical analyses were conducted using Jamovi version 2.3.0 and normality distribution was assessed for baseline quantitative data by means of Shapiro-Wilk tests. A p value threshold of 0.05 was considered statistically significant for all analyses. Values presented in the results section represent mean±standard deviation. In the second study, urinary TNF-α concentrations, pain intensity ratings and disability scores at baseline and after eight sessions of SMT were compared using one-tailed paired t-tests for normally distributed data and the Wilcoxon Rank-Sum for non-normal distributions. Effects sizes are reported as Cohen's d for the t-tests and rank biserial correlations for the Wilcoxon Rank-Sum. In this study, for rank biserial correlations, effect sizes are interpreted as small for r=0.2, medium for r=0.5, and large effect sizes for r=0.8.

Kruskal-Wallis analyses of variance (ANOVA) were conducted using these categorical variables as between-groups factors to identify potential differences in urinary concentrations of TNF-α at baseline, according to sex, pain trajectories and the presence of comorbidities as data were not normally distributed. Spearman rank correlation coefficients were calculated to examine the associations between baseline values of TNF-α, the number of years with CLBP, pain intensity and disability. Follow-up and percent-changes in TNF-α values were identified as predictors in simple regression models with follow-up and percent change values in pain intensity and disability as dependent outcomes.

Patients reported current pain intensity verbally using a Numerical Rating Scale from 0 to 10 (NRS-11), in which 0 signified the absence of pain and 10 the worst possible pain for the patient. Functional disability due to chronic low back pain was measured using the validated version of the Oswestry Disability Index (ODI).

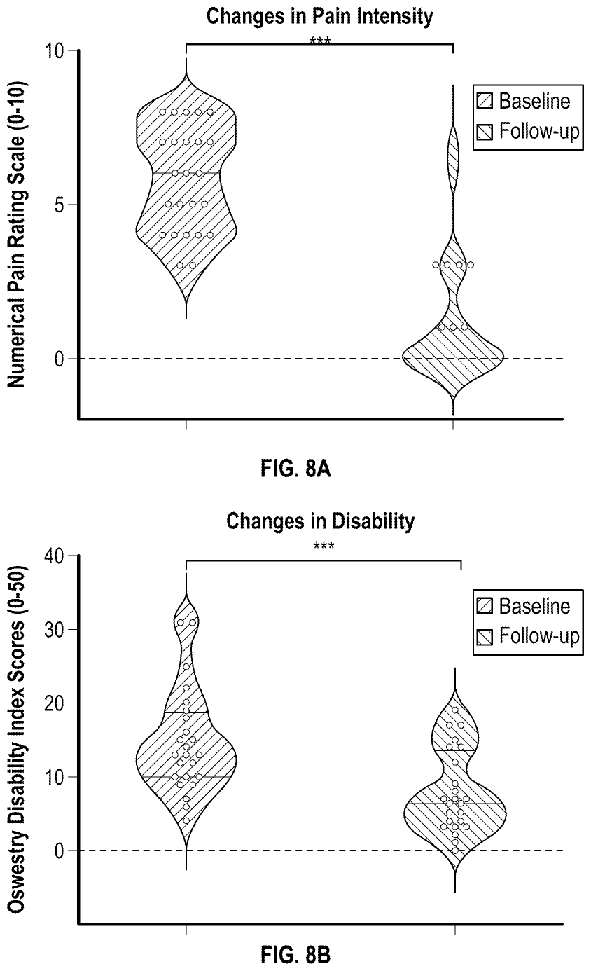

In the second investigatory study, twenty-four patients (18 women and 6 men) suffering from low back pain met the inclusion and exclusion criteria, were accepted to participate in the second investigatory study by providing PROMs and urine samples and concluded the treatment period. Referring to FIG. 6, FIG. 8A and FIG. 8B, pain intensity and disability data were normally distributed (W=0.917 and W=0.930 respectively, both p>0.05) in this second study, with significant reductions observed in clinical outcomes following the eight sessions of SMT. Pain intensity was reduced in 4.6±2.1 points in the 0-10 NRS scale, p<0.001, Cohen's d=2.2 (FIG. 6 and FIG. 8A). Furthermore, the degree of disability caused by CLBP was also reduced in 6.9±5.5 points in the ODI 0-50 scale, p<0.001, Cohen's d=1.24 (FIG. 6 and FIG. 8B).

The second investigatory study corroborates the results of the first investigatory study of elevated levels of TNF-α in urine samples of patients with chronic low back pain. Furthermore, in the cohort of patients in the second investigatory study, urinary concentrations of this TNF-α cytokine were responsive to conservative care based on instrument assisted SMT. Different pain trajectories showed significant differences in baseline levels in urinary TNF-α, the highest levels being measured in patients with unremitting pain. In turn, the second investigatory study demonstrates that baseline TNF-α levels can be used to predict changes in pain intensity and follow-up values in disability, while fluctuations in urinary TNF-α levels can be used to predict changes in both pain intensity and disability scores. Overall, the results of the second investigatory study corroborate the contemplated use of measured urinary TNF-α levels as a biomarker for the recovery from lower back pain episodes or even recurrent lower back pain.

In the absence of inflammation, TNF-α levels are presumed to approach zero, with minimal fluctuations. A mean serum value of 1.51 pg/mL and an upper bound of 2.53 pg/ml (95th percentile) are suggested to rule out systemic inflammation. Reference values in urine samples were found to be 0.4±0.9 pg/ml, which supports the findings of elevated TNF-α levels in the patient group of the second investigatory study. However, the values of the measured urinary TNF-α levels differed between patients with different pain trajectories, specifically between patients with ‘ongoing’ compared to ‘episodic’ pain trajectories. Participants categorized as ‘ongoing’ had generally higher urinary levels of TNF-α (6.6±4.6 pg/mg), followed by patients classified as ‘fluctuating’ (2.7±4.2 pg/mg), and finally, patients with ‘episodic’ chronic low back pain, which had undetectable levels of this cytokine, making them biochemically identical to healthy individuals in this aspect. Previous assessment of urinary TNF-α values in chronic low back pain patients in the first investigatory study showed mean values of 6.0±7.0 pg/mg, in a cohort where 75% of patients were classified as ‘ongoing’, which is consistent with the data derived in the second investigatory study.

The second investigatory study demonstrates that measured levels of urinary TNF-α levels can act as a patient stratification biomarker, which is crucial in health conditions with heterogeneous pathophysiology, such as pain. This use of such measured urinary TNF-α level can be used to discriminate patients with chronic low back pain according to their pain trajectory. Particularly, patients with persistent pain (whether ongoing or fluctuating, but not remitting), can be better identified by this stratification biomarker.

The second investigatory study also demonstrates that measured levels of urinary TNF-α levels can act as a patient stratification biomarker that is indicative of recovery or to predict the response to treatment. For example, the second investigatory study demonstrates that urinary TNF-α levels (acting as a biomarker) can be used to assess the response to manual therapy in chronic low back pain patients. In the second study, baseline values in urinary TNF-α values explained 20.7% of the variance in changes in pain intensity and 40.9% of the variance in disability after treatment. Similarly in the second study, percent change in TNF-α predicted 68% and 34% of the changes in pain intensity and disability scores respectively, indicating that urinary TNF-α values can provide a reliable objective measure of response to treatment, i.e., a reduction in TNF-α levels may be indicative of recovery from episodes of lower back pain and permanently elevated levels would be associated with a lack of recovery or with persistent chronic low back pain symptoms with minor or major fluctuations but without long pain-free period. Thus, it is contemplated that patients with ongoing pain have higher levels of TNF-α compatible with no recovery, while patients with episodic chronic low back pain would show the lowest levels, as they have the capacity of recovering from an episode.

In another aspect, it is an object of the invention to provide kits and methods for evaluating the relative level of the TNF-α cytokine in a patient. According to this object, the invention provides a kit which contains at least one cytokine-specific detection reagent that is adapted to detect a threshold level of TNF-α cytokine, which threshold level correlates with a heightened level of TNF-α cytokine in the patient. In one exemplary embodiment, the cytokine specific reagent is specific for TNF-α and the reagent can comprise an antibody or antibody fragment.

Also, according to this aspect of the invention, a method of assessing the heightened level of TNF-α cytokine in the patient is provided. The identification of the heightened level of the TNF-α cytokine in the patient can be used as a guide in chiropractic care, as it can be used to monitor the efficacy of ongoing chiropractic treatment plans and/or protocols for a respective patient. This method entails comparing the amount of TNF-α cytokine in a patient urine sample with a threshold level, thereby gauging the inflammatory state of the patient. In this embodiment, the cytokine specific reagent is specific for TNF-α and the reagent may comprise an antibody or antibody fragment.

It is another object of the invention to provide an improved method of treating chronic low back pain patients. Further to this object, a method is provided where a patient is administered an effective amount of SMT-induced modulation and the level of the TNF-α cytokine in the patient is compared with a threshold level prior to the initiation of a subsequent SMT-induced modulation, which is typically spaced at least a day from the prior SMT-induced modulation. In one embodiment, the method involves using the threshold level to guide treatment, so that when the threshold is crossed, treatment is continued as the heightened level of TNF-α is indicative of an undesirably high level of inflammatory response. Treatment would be continued until the threshold is no longer approached or crossed.

It is contemplated in this aspect of the invention that threshold levels of TNF-α cytokine in the patient suffering from chronic low back pain can be used to guide the health care professional in using chiropractic therapies. In particular, these threshold levels provide a marker, indicating whether or not the applied respective chiropractic therapies are having an efficacious and positive physical result on the patient. In this aspect, a common application is in monitoring chiropractic therapies, where the subject threshold levels are used to decide whether a patient is improving from the one applied chiropractic therapy, such as a SMT-induced modulation, and/or requires additional applications of applied chiropractic therapy, such as a SMT-induced modulation.

In a broad sense, a threshold level may be a level that is found in a patient suffering from chronic low back pain, with any deviation associated with inflammatory response being indicative of that state. In the present aspect, it is contemplated that the threshold would represent a minimum level, below which chiropractic therapy could be reduced or halted. In various optional aspects, the minimal threshold levels of TNF-α cytokine concentration in the sample can be at least about 6 pg/ml, optionally at least about 7 pg/ml, and optionally at least about 8 pg/ml. In this context, for example and without limitation, a threshold level of TNF-α cytokine concentration in the sample for a patient suffering from chronic lower back pain is meet when the TNF-α cytokine level is within at least about ±15% of the threshold number, but preferably is within at least about ±10% of the threshold. Further in this context, without limitation, the urine sample volumetric sample size is uniform and, as discussed below, the urine sample is normalized via addition of water as required.

In an optional aspect, the minimal threshold levels of TNF-α cytokine concentration in the sample can be at least about 4 pg/mg, optionally at least about 5 pg/mg, optionally at least about 6 pg/mg, and optionally at least about 7 pg/mg. In this context, for example and without limitation, a threshold level of TNF-α cytokine concentration in the sample for a patient suffering from chronic lower back pain is meet when the TNF-α cytokine level is within at least about ±15% of the threshold number, but preferably is within at least about ±10% of the threshold.

In yet another optional aspect, the minimal threshold levels of TNF-α cytokine concentration in the sample can be set to capture a lower level of TNF-α cytokine concentration in the sample as this lower can be indicia of episodic lower back pain rather than chronic low back pain. In this context, it is contemplated that the minimal threshold levels of TNF-α cytokine concentration in the sample can be at least about 1.5 pg/mg, optionally at least about 2.0 pg/mg, optionally at least about 3.0 pg/mg, and optionally at least about 3.5 pg/mg. In this context, for example and without limitation, a threshold level of TNF-α cytokine concentration in the sample for a patient suffering from episodic lower back pain is meet when the TNF-α cytokine level is within at least about ±15% of the threshold number, but preferably is within at least about ±10% of the threshold.

In embodiments, it is contemplated that threshold levels of TNF-α cytokine in the patient suffering from chronic low back pain can be used provide a marker, indicating whether or not the applied respective chiropractic therapies are having an efficacious and positive physical result on the patient. In this aspect, a common application is in monitoring chiropractic therapies, where the subject threshold levels are used to decide whether a patient is improving from the one applied chiropractic therapy, such as a SMT-induced modulation, and/or requires additional applications of applied chiropractic therapy, such as a SMT-induced modulation.

In this aspect, the threshold level in this aspect can be a level that is found in a patient suffering from chronic low back pain, with any deviation associated with inflammatory response being indicative of that state. In the present aspect, it is contemplated that the threshold would represent a minimum level, below which chiropractic therapy could be reduced or halted. In various optional aspects, the TNF-α cytokine concentration in the sample can be least about 1.5 pg/mg, optionally at least about 2.0 pg/mg, optionally at least about 4 pg/mg, optionally at least about 5 pg/mg, optionally at least about 6 pg/mg, and optionally at least about 7 pg/mg. Further in this context, without limitation, the urine sample volumetric sample size is uniform and the urine sample may or may not be normalized via addition of water as required. In this context, for example and without limitation, a threshold level of TNF-α cytokine concentration in the sample for a patient suffering from chronic lower back pain is meet when the TNF-α cytokine level is within at least about ±15% of the threshold number, but preferably is within at least about ±10% of the threshold. In this aspect, it is contemplated that a colorimetric analysis of the test result would provide guidance as the level of TNF-α cytokine that is present in the subject urine sample, i.e., the more the test changes or deepens in color, the higher the level of TNF-α cytokine that is present.

In exemplary lateral flow assay models, it is contemplated that a colorimetric analysis of the depth and saturation of the color change resulting from the presence of TNF-α in the patient's tested urine sample can provide clinical assessment of the respective threshold of TNF-α present. For example, if the color of the tested sample changes fractionally, it would be indicative of a minimal level of detected TNF-α, which can be indicia of episodic lower back pain rather than chronic low back pain. Whereas, if the color of the tested sample changes significantly, it would be indicative of a higher level of detected TNF-α, which can be indicia of chronic low back pain rather than episodic lower back pain.

In one aspect, the kits according to the invention can typically comprise at least one cytokine-specific detection reagent. Optionally, it is contemplated that the kits can contain at least two cytokine-specific detection reagents. In most cases, each reagent will be adapted to detect a threshold level of TNF-α cytokine, which correlates with the inflammatory state of a patient. In one aspect, the kit for can be used to assess the inflammatory state of a patient via the assessment of the level of TNF-α cytokine that is present in the patient's urine, which is useful in guiding the physician in choosing an optimal treatment regimen. As such, the kits can be applied, for example, to monitor chiropractic treatments, to monitor efficacy of chiropractic treatments and to monitor ongoing inflammatory response in patients suffering from chronic low back pain.

Some embodiments of the present kits contain the detection reagent in association with a suitable testing substrate. Suitable substrates include “dipsticks,” test-strips, microtiter plates, microtubes, and the like. The described kits generally implement the methods, described below, and should be read in that context.

The cytokine-specific detection reagent of the kit generally confers the ability to detect specifically TNF-α, the cytokine of interest, like those mentioned above, in some cases quantitatively. Typically, this reagent will be able to bind specifically to TNF-α, and will be detectable, directly or indirectly. For instance, the reagent may be an antibody, and may comprise a detectable label, such as a radionuclide, an enzyme, or a fluorescent tag. The label may be detected, for example, using conventional immunoassays, which include enzyme-linked immunosorbent assays (ELISAs), radioimmunoassays (RIAs), and the like.

Due to their recognized ability to bind specifically and to their ease of production, antibodies are contemplated as a means of conferring the cytokine-binding ability of the detection reagent. Antibodies include, but are not limited to polyclonal antibodies, monoclonal antibodies (mAbs), humanized or chimeric antibodies, single chain antibodies including single chain Fv (scFv) fragments, Fab fragments, F(ab′)₂ fragments, fragments produced by a Fab expression library, epitope-binding fragments, and multivalent forms of any of the above.

In general, techniques for preparing polyclonal and monoclonal antibodies as well as hybridomas capable of producing the desired antibody are well known in the art. Antibody fragments include any portion of the antibody which includes the paratope and is capable of binding a cytokine of interest. Antibody fragments specifically include F(ab′)₂, Fab, Fab′ and Fv fragments. These can be generated from any class of antibody, but typically are made from IgG or IgM. They may be made by conventional recombinant DNA techniques or, using the classical method, by proteolytic digestion with papain or pepsin. F(ab′)₂ fragments are typically about 110 kDa (IgG) or about 150 kDa (IgM) and contain. two antigen-binding regions, joined at the hinge by disulfide bond(s). Virtually all, if not all, of the Fc is absent in these fragments. Fab′ fragments are typically about 55 kDa (IgG) or about 75 kDa (IgM) and can be formed, for example, by reducing the disulfide bond(s) of an F(ab′)₂ fragment. The resulting free sulfhydryl group(s) may be used to conveniently conjugate Fab′ fragments to other molecules, such as localization signals.

Fab fragments are monovalent and usually are about 50 kDa (from any source). Fab fragments include the light (L) and heavy (H) chain, variable (VL and VH, respectively) and constant (CL CH, respectively) regions of the antigen-binding portion of the antibody. The H and L portions are linked by one or more intramolecular disulfide bridges.

Fv fragments are typically about 25 kDa (regardless of source) and contain the variable regions of both the light and heavy chains (VL and VH, respectively). Usually, the VL and VH chains are held together only by non-covalent interactions and, thus, they readily dissociate. They do, however, have the advantage of small size and they retain the same binding properties of the larger Fab fragments. Accordingly, methods have been developed to crosslink the VL and VH chains, using, for example, glutaraldehyde (or other chemical crosslinkers), intermolecular disulfide bonds (by incorporation of cysteines) and peptide linkers. The resulting Fv is now a single chain (i.e., scFv). Single chain antibodies are formed by linking the heavy and light chain fragments of the Fv region via an amino acid bridge, resulting in a single chain FV (scFv).

It is contemplated that the kits contain at least one cytokine-specific reagent that is specific for TNF-α. In one aspect of the invention, the reagent can comprise an enzyme-linked antibody or antibody fragment.

The kits of the invention are configured to detect a specific or general threshold of TNF-α, which correlates with the inflammatory state of the patient. Such thresholds, and their determination, are detailed below. For convenience, it is advantageous to adapt the cytokine-specific detection reagent(s) to detect a certain minimal threshold. In this way, a “yes” or “no” answer can be provided, generally indicating whether the patient has a heightened level of TNF-α, which correlates to a heighted inflammatory response. Thus, for example, colorimetric detection might be employed, whereby the presence of color indicates that a threshold level, correlating with the inflammatory state, has been reached. Further, the degree of color saturation of the test sample can indicate the respective level of the TNF-α that is present in the sample.

Typically, the reagents of various assays (e.g., ELISAs, RIAs, and the like) will be able to detect levels of the target cytokine(s) that are lower than the threshold, i.e., they are more sensitive than they need to be. The artisan will be well-aware of methods of reducing the sensitivity of the present systems in order to provide a signal at a given threshold level. A particularly useful kit will include a reagent system that can provide a “yes” or “no” answer as to whether a patient is still suffering from a heightened inflammatory response via the sensing of a threshold level of TNF-α cytokine present in the patient's urine and further interventional chiropractic treatment therapy is desired.

The kits may be adapted for private to commercial-scale use, for the convenience of the individual clinician, the clinical research center and even commercial diagnostic laboratories. For example, in a private clinical setting, a “dipstick”-type arrangement may be convenient. In one aspect, the cytokine-specific detection reagent may come applied to the dipstick. Thus, the kit may be used by contacting a patient sample to the dipstick-associated reagent. The detection reagent may then be visualized using conventional colorimetric means, for example. Of course, another arrangement may call for contacting the sample with the dipstick, and then application of the cytokine-specific detection reagent; the exact arrangement is a matter of choice.

Optionally, prior to testing the patient urine sample with the kit, it may be desired to ensure that the collected urine sample is normalized. In this aspect, the urine sample can be normalized to a specific gravity, such as for example and without limitation, a specific gravity of about 1.002, which value may require dilution of the urine sample with water.

As described, it is contemplated that the general method of the invention allows for the assessment of the inflammatory state of the patient via analysis of a patient urine sample that is collected in a sample collection device, such as, for example and without limitation, a selectively sealable container, cup or the like. The basic method comprises comparing the amount of TNF-α in the collected sample to a threshold level.

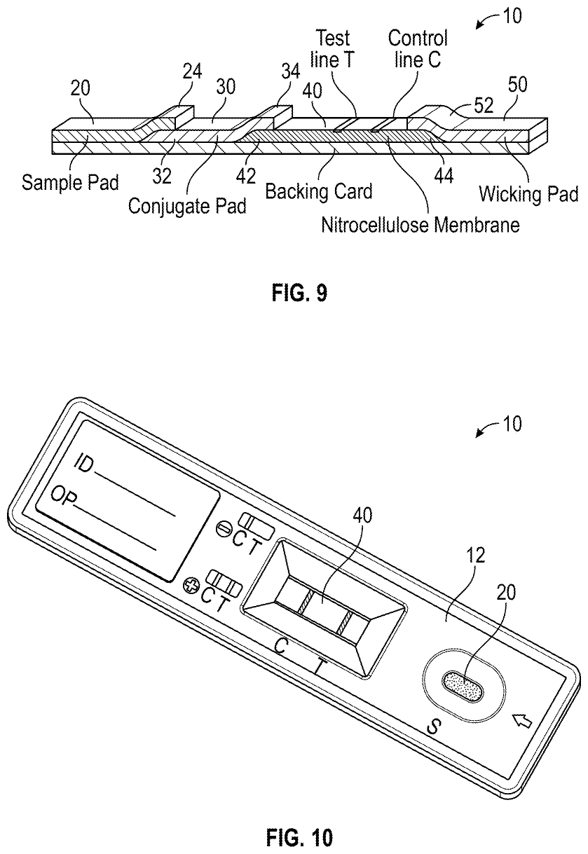

Referring to FIG. 9, an exemplary kit is shown that uses a lateral flow modality, which can be used as a point of care (POC) diagnostic kit. In embodiments, the TNF-α detection kit 10 includes a plurality of: a sample pad 20, a conjugate pad 30; a reagent membrane 40, and a wicking pad 50. As shown, the sample pad 20 is configured to absorb a urine sample and to control the delivery of portions of the urine sample to the conjugate pad. The conjugate pad 30 is formed of a medium that is configured for holding at least one dried nanoparticle-antibody conjugate. In operation, after introduction of a patient's urine thereto, the at least one dried antibody-nanoparticle conjugate is configured for controlled release of re-solubilized conjugate onto the reagent membrane. The reagent membrane 40 can be, without limitation, a nitrocellulose membrane or the like, which is configured to provide a solid phase for immobilizing respective test and control line reagents. Also shown is a wicking pad 50 that is configured to pull the urine sample through the test strip at a desired rate. In operation, the wicking pad is configured to provide uniform capillary flow through the reagent membrane, as it absorbs the applied urine sample and further can help to prevent back flow of the urine sample.

As shown, a distal portion 24 of the sample pad overlaps a proximal portion 32 of the conjugate pad and a distal portion 34 of the conjugate pad overlaps a proximal portion 42 of the reagent membrane. Further, a proximal portion 52 of the wicking pad overlaps a distal portion 44 of the regent membrane All of these components can be mounted onto a backing card. As one will appreciate, the controlled overlap between all these components allows the patient urine sample to move through the test strip via capillary action.

In aspects, and referring to FIG. 10, the test component components can be housed inside of a cassette 12, such as the exemplified polymer housing, so that only the sample pad and reagent membrane with the test and control lines (test line (T) and control line (C)) are visible to the end user through spaced openings defined in the surface of the cassette 12. In operation, portions of the urine sample, which may include a few drops of the subject patient's urine, can be applied to the sample pad and, after a predetermined period of time, the presence or absence of a colorized test line indicates the presence or absence of the TNF-α in the tested urine. In aspects, the applied urine sample can be, but is not required to be, a first sample of the day urine sample from the patient.

Further, in embodiments, the predetermined period of time to indicate the presence or absence of TNF-α can be a time period that is less than 5 minutes, less than 10 minutes; less than 15 minutes, less than 20 minutes, less than 25, and/or less than 30 minutes. Still further, the predetermined period of time can be a time period that is between 2 to 60 minutes, between about 3 to 30 minutes, between about 5 to 25 minutes, and/or between 10 to 20 minutes.

As contemplated, the conjugate pad can be configured to contain conjugates, which are also called detector nanoparticles or probes. In aspects, the nanoparticles can be functionalized antibodies anti-TNF-α, which recognize TNF-α. In embodiments, the test line on the reagent pad can be configured to contain immobilized antibodies anti-TNF-α that are configured to operatively bind to TNF-α to show a positive or negative result. This showing of a positive result can be a colorized response resulting from the binding of the antibodies anti-TNF-α to TNF-α present in the urine sample. In aspects, and as described above, the saturation of the color change noted in the test line could be used as an indication of the relative level of TNF-α in the urine sample.

In this aspect, the control line can be configured to contain an immobilized antibody that will bind to the antibody on the surface of the particle whether or not the analyte is present, to confirm that the assay is working correctly. In embodiments, the control line can be a secondary antibody specific to the host species of the primary antibody on the conjugate.

In operational aspects, the patient urine sample can be added to the sample pad whereupon it will be drawn through the TNF-α detection kit 10 by capillary action. In operation, the sample pad 20 can filter unwanted portions of the sample (such as red blood cells, solid particulates, and the like) and can, in aspects, normalize the pH of the sample if needed before the urine sample reaches the conjugate matter in the conjugate pad, such that the urine sample can be “normalized” to overcome sample-to-sample variability and ensure accurate and reproducible results.

Subsequently, the urine sample wicks to the conjugate pad that contains dried nanoparticle conjugate, which are then solubilized on contact with the aqueous urine sample and can operatively bind to TNF-α (if present). The nanoparticles and urine sample continue to flow through the nitrocellulose membrane until they reach the test line and control line. As described above, binding events at the test line provide a visual indication of whether or not TNF-α was present in high enough quantities to be detected. In one aspect, and without limitation, the test line can be configured to provide a visual indication of whether or not the detected level of TNF-α exceeded at least the minimal threshold level of TNF-α or, optionally, exceeds a predetermined threshold level of TNF-α.

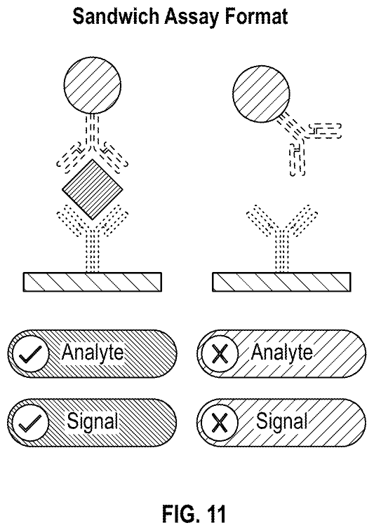

In embodiments, it is contemplated that the TNF-α lateral flow detection kit is a lateral flow assembly that follows the general form of a sandwich assay. Referring to FIG. 11, such a sandwich assay format provides for the detection of TNF-α, which is a relatively large analyte. As the analyte has at least two distinct binding sites (i.e., epitopes), the illustrated sandwich assay uses an antibody, to an epitope of TNF-α, conjugated to a nanoparticle and another, different, antibody to another epitope of TNF-α immobilized on the text line T. If TNF-α is present in the urine sample, TNF-α will bind the nanoparticle conjugate to the test line T, yielding a positive signal. As one will appreciate, the sandwich format results in a signal intensity that is proportional to the amount of analyte present in the sample. In operation, and to verify that the assay runs correctly and indicates a valid result, the control line (C), which is immobilized on the reagent membrane located proximate to and, in one exemplary aspect, downstream of the test line C, is typically a species-specific secondary antibody that can bind to the antibody that is conjugated to the nanoparticle probe. In embodiments, the control line C should be visible whether or not TNF-α is present in the sample.

In embodiments and for example and without limitation, for the conjugate pad, gold nanoparticles can be used and the antibodies can be attached to them.

In embodiments, one exemplary method of conjugation is passive conjugation. In embodiments, a passive adsorption process relies on interaction forces like van der Waals forces and other weak attractions that exist between the macromolecule, such as an antibody or protein, and the surface of the nanoparticle. The resultant bonding between the antibody and the nanoparticle probe is influenced by the properties of both the nanoparticle's surface and the surrounding environment for coupling. In situations involving less hydrophobic antibodies or a more hydrophilic nanoparticle surface (for example, one modified with —COOH groups), attachment can occur through a combination of ionic and hydrophobic interactions. Even slight variations in pH levels can modify the dynamics of association and impact the efficiency of conjugation.

To identify the optimal conditions for antibody adsorption, it is advisable to conduct a pH titration and assess the antibody-to-particle ratio. Typically, it is recommended to maintain the pH of the adsorption buffer slightly above the protein's isoelectric point, which can vary from one antibody to another. Notably, the Fc region of the antibody, being generally more hydrophobic, tends to be more prone to adsorption compared to the Fab region, offering some degree of control over binding orientation. Typically, a substantial excess of antibody relative to the nanoparticle surface area is employed to ensure a densely packed surface binding and robust salt stability of the gold nanoparticles post-conjugation.

In embodiments, another exemplary method of conjugation is covalent coupling, which is stable with less antibody desorption and requires fewer antibodies during conjugation. Covalent coupling or attachment can be accomplished with several different types of chemistry. In one example, and without limitation, an amide bond can be formed to connect nanoparticle to antibody, which would require the nanoparticle to be previously functionalized through the addition of carboxylic acid surfaces to allow formation of the covalent bond with free amines on the antibody.

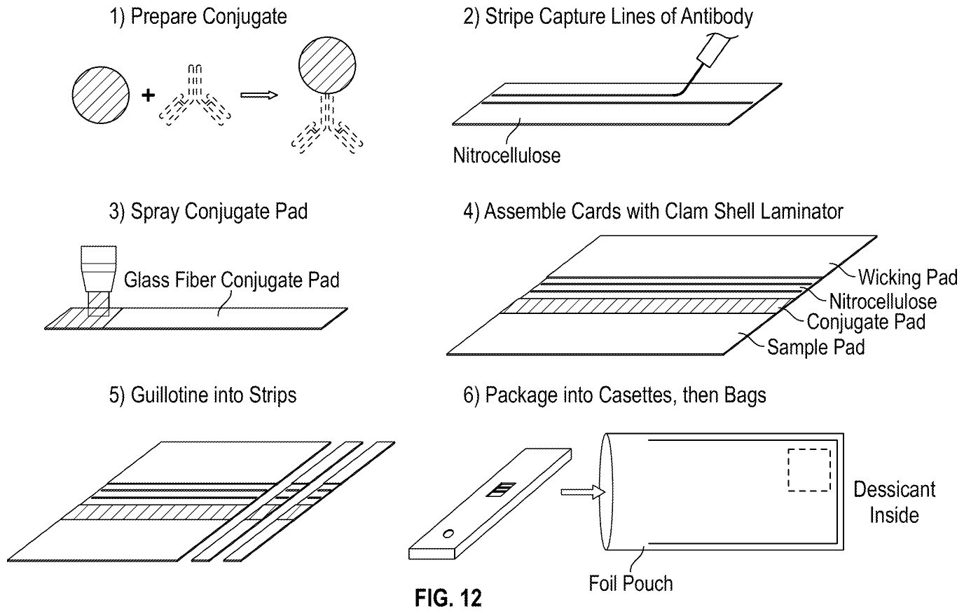

Referring to FIG. 12, in embodiments for the preparation of a lateral flow TNF-α lateral flow detection kit cassette of the previously described exemplary method of passive conjugation, in an initial step 1, conjugates (nanoparticle+antibody) are prepared and subsequently transferred to an appropriate solution for spraying and drying onto the conjugate pad.

Subsequently, in step 2, a dispenser with tube, such as a flexible hollow glass, can be connected to a syringe pump for reproducible striping of test and control lines. The conjugate can be applied to the conjugate pad, such as a glass fiber pad, in step 3. Preferably, the conjugate can be applied to the conjugate pad using a non-contact spray head on the dispenser. Once the solutions are applied to both the reagent membrane and the conjugate pad, these components are dried and cured in an oven at a suitable temperature, such as between about 20-50° C.

After drying, the reagent membrane, the sample pad 20, the conjugate pad 30; the reagent membrane 40, and the wicking pad 50 can be transferred to a dry room having less than about 30%, 20%, and/or 10% relative humidity for assembly onto a backing card, such as the exemplified adhesive backing card, using a clamshell laminator. In aspects, the laminator ensures the accurate placement of components onto the adhesive backing card with the correct overlaps and controlled application pressure. These fully laminated backing cards are referred to as “master cards.” As shown in step 4, such a master card can be operatively assembled by applying a nitrocellulose membrane first, followed by the conjugate pad, wick pad, and subsequently the sample pad. Next, in step 5, the assembled master cards can be cut into individual strips that are appropriately sized for the cassette with an automated guillotine. Finally, the sized strips are inserted into the interior chamber of the cassette and the formed TNF-α lateral flow detection kit cassette can be sealed in a foil pouch with a desiccant.

In embodiments, in step 1 where conjugates (nanoparticle+antibody) are prepared, the “uncoated” nanoparticle surfaces can be secured with citrate molecules to form relatively loose bonds, which allows them to be readily displaced when TNF-α antibodies are introduced. Over time, the protein can gradually attach itself to the nanoparticle surface through relatively feeble interactions, such as van der Waals forces and/or ionic attractions. In the case of covalent coupling, carboxyl groups on the nanoparticle surface will be covalently linked to TNF-α antibody with a primary amine via carbodiimide crosslinker chemistry. This chemistry produces a stable amide or peptide bond between the nanoparticle surface and the antibody resulting in a robust conjugate. In various embodiments, it is contemplated that the nanoparticle can comprise a gold nanosphere have a diameter of between about 30 to 170 nm, between about 40 to 150 nm; preferably between about 50 to 120 nm, more preferably between about 70 to 100 nm, and most preferred about 80 nm.

In embodiments, two antibodies can be used that can both bind to the TNF-α. The first antibody can be a mouse anti-human TNF-α monoclonal antibody conjugated with the gold nanoparticles and another antibody that is configured to recognize a different epitope of TNF-α, which would be immobilized in the test line.

In embodiments, to achieve the passive adsorption of anti-TNF-α antibodies onto nanoparticles, the buffer unitized should not contain any extra stabilizing proteins (such as BSA and the like) and salt preservatives (such as sodium azide and the like) and the pH level should be further optimized in the buffer to enhance the effectiveness of the conjugation process. It is contemplated that the pH of the buffer will be maintained (using HCl/NaOH) from between 3.5 to 9.5 pH, from between 4.0 to 9.3 pH, from 5 to 9 pH, and/or between about 6-8 pH). The stability of colloidal solution and polydisperse can use the calculated values from the ratio of absorbance at λmax/580 nm and 600 nm/λmax (λmax=520 nm) after adding of NaCl, respectively. As appreciated, the desired pH selected to the buffer is the most stable and the generates the least amount of polydispersion of nanoparticles.

For the conjugation of antibodies to nanoparticles, it is desired critical that the antibody is in the correct buffer as described above. In embodiments, the antibody for conjugation can be purified and adjusted to a concentration of at least 1 mg/mL, at least 3 mg/mL, at least 5 mg/mL, and/or at least 10 mg/mL in a low ionic strength buffer. As described, antibodies can be purified and transferred into an amine-free buffer using spin columns or dialysis tubing with the appropriate molecular weight cut-off.

In an additional embodiment and to purify antibodies from additional stabilizing proteins, an affinity column such as a protein A or G column can be used. Since most conventional protocols for separation with affinity columns use Tris as a buffer, subsequent purification may be desired to remove free amines after the antibody is recovered. After protein purification, the concentration of the antibody can be verified to ensure that the correct amount of antibody is being conjugated to the nanoparticle. For measuring protein concentration, a BCA assay, a Bradford assay, or the like can be used.

In aspects, the control line on the reagent membrane functions as an internal quality or procedural control and can will be visible in the presence or absence of TNF-α to show or otherwise indicate to the end user that the assay is functional and that the results are valid. The control line antibody can be a goat anti-mouse polyclonal antibody, specific to the species of the conjugated antibody. In this exemplary system, the secondary antibody can bind the conjugated antibody in the presence or absence of TNF-α and result in a visual readout.

For sandwich assays, the striping of the control and test lines can utilize 1 mg/mL is a recommended starting point for test and control line antibody concentrations but can range from about 0.5 to 2 mg/mL. As contemplated, the respective selected concentration can depend on the sensitivity requirements and the affinity of the antibodies to the analyte in the sample.

In embodiments, for each strip that is formed from the noted master cards, it is contemplated that each strip will be contain about 0.1 to 20 microliters/strip of nanoparticles; preferably about 0.2 to 10 microliters/strip of nanoparticles; and more preferably about 0.5 to 5 microliters/strip of nanoparticles. In additional embodiments, for nanoparticles, it is contemplated that the concentration range of the antibody used to cover the nanoparticles would be about 0.1 to 10 mg, preferably about 0.2 to 5 mg, and more preferred about 0.5 mg.

In embodiments, for the test line, it is contemplated that the concentration range of the antibody used in the test line would be about 0.1 to 10 mg/ml, preferably about 0.2 to 5 mg/ml, and more preferred about 0.5 to 2 mg/ml. Similarly, for the control line, it is contemplated that the concentration range of the antibody used in the test line would be about 0.1 to 10 mg/ml, preferably about 0.2 to 5 mg/ml, and more preferred about 0.5 to 2 mg/ml.

Although only a few exemplary embodiments have been described in detail herein, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims.

What is claimed is:

1. A diagnostic kit comprising:
a sample collection device configured to obtain a urine sample from a patient suffering from lower back pain;
a detection kit comprising a binding agent for detecting TNF-alpha cytokine in the urine sample, wherein the detected TNF-alpha cytokine is associated with an inflammatory response in the patient that is indicative of a lower back condition; and
written instructions for use.

2. The kit of claim 1, wherein the detection kit is a point of care (POC) diagnostic kit.

3. The kit of claim 2, wherein the POC diagnostic kit is a lateral flow immunoassay.

4. The kit of claim 2, wherein the POC diagnostic kit is a colorimetric lateral flow immunoassay.

5. The kit of claim 1, wherein the lower back condition is chronic low back pain.

6. The kit of claim 1, wherein the detected TNF-alpha cytokine exceeds a minimal concentration level for the detection kit to test positive, and wherein the detection kit will test positive if the level of detected TNF-alpha cytokine is within at least about ±15% of the minimal concentration level.

7. The kit of claim 6, wherein the minimal concentration level of detected TNF-alpha cytokine is at least about 6 pg/ml.

8. The kit of claim 6, wherein the minimal concentration level of detected TNF-alpha cytokine is at least about 4 pg/ml.

9. The kit of claim 6, wherein the minimal concentration level of detected TNF-alpha cytokine is at least about 1.5 pg/ml.

10. The kit of claim 1, wherein the detection kit comprises:
a sample pad configured to absorb a portion of the urine sample;
a conjugate pad in fluid communication with the sample pad, the conjugate pad being configured for holding at least one dried nanoparticle-antibody conjugate;
a reagent membrane in fluid communication with the conjugate pad, the reagent membrane having a test line and a spaced control line, the test line being configured to provide a solid phase for immobilizing a text line reagent and the control line being configured to provide a solid phase for immobilizing a text line reagent; and
a wicking pad in fluid communication with the reagent membrane and configured to provide uniform capillary flow through the reagent membrane.

11. The kit of claim 10, wherein the conjugate pad is configured to contain nanoparticles, and wherein the nanoparticles can be functionalized antibodies anti-TNF-α, which recognize TNF-α.

12. The kit of claim 11 wherein the test line on the reagent pad is configured to contain immobilized antibodies anti-TNF-α that are configured to operatively bind to TNF-α to show a positive result.

13. The kit of claim 12, wherein the showing of a positive result is a colorized response resulting from the binding of the antibodies anti-TNF-α to TNF-α present in the urine sample.

14. The kit of claim 12, wherein the test line is configured to provide a visual indication of whether or not the detected level of TNF-α exceeds a predetermined level of TNF-α.

15. The kit of claim 1, wherein the presence of TNF-α in the urine sample will be indicated within a time period of between about 2 to 60 minutes.

16. A method of assessing a heightened level of TNF-α cytokine in a patient suffering from chronic low back pain, comprising:
obtaining a urine sample from the patient;
measuring the level of TGF-α cytokine thereof in the urine sample using a kit comprising at least one cytokine-specific detection reagent that is configured to detect a threshold level of TNF-α cytokine, which threshold level correlates with a heightened level of TNF-α cytokine in the patient; and
comparing the level of the TGF-α cytokine to a predetermined threshold level to gauge an inflammatory state of the patient.

17. The method of claim 16, wherein the cytokine specific reagent is specific for TNF-α.

18. The method of claim 17, wherein the cytokine specific reagent comprises an antibody or antibody fragment.

19. The method of claim 16, wherein the threshold level provides a marker that indicates whether or not an applied respective chiropractic therapy is having an efficacious result.

20. A method of adjusting a chiropractic treatment protocol to be administered to a patient suffering from chronic lower back pain, comprising:
obtaining a urine sample from the patient;
measuring the level of TGF-α cytokine thereof in the urine sample;
comparing the level of the TGF-α cytokine to a predetermined threshold level; and
administering desired protocols of chiropractic treatment to the patient on the comparison of TGF-α cytokine levels.

21. The method of claim 20, wherein the step of measuring uses a kit comprising at least one cytokine-specific detection reagent that is configured to detect a threshold level of TNF-α cytokine, which threshold level correlates with a heightened level of TNF-α cytokine in the patient.

22. The method of claim 21, wherein the cytokine specific reagent is specific for TNF-α.

23. The method of claim 22, wherein the cytokine specific reagent comprises an antibody or antibody fragment.

24. The method of claim 20, wherein the threshold level provides a marker that indicates whether or not an applied respective chiropractic therapy is having an efficacious result.
